(19) United States
(12) Patent Application Publication
(10) Pub. No.: US 2017/0189864 A1
(43) Pub. Date: Jul. 6, 2017

(54) SELECTIVE PHENOL REMOVAL MEMBRANES AND VALORIZATION OF OLIVE OIL WASTE STREAMS

(71) Applicant: B.G. NEGEV TECHNOLOGIES AND APPLICATIONS LTD., AT BEN-GURION UNIVERSITY, Beer-Sheva (IL)

(72) Inventors: Jack GILRON, Beer-Sheva (IL); Charles LINDER, Rehovot (IL); Zeev WIESMAN, Lehavim (IL)

(73) Assignee: B.G. NEGEV TECHNOLOGIES AND APPLICATIONS LTD., AT BEN-GURION UNIVERSITY, Beer-Sheva (IL)

(21) Appl. No.: 15/313,743

(22) PCT Filed: May 27, 2015
(86) PCT No.: PCT/IL2015/050547

§ 371 Date: Nov. 23, 2016

Related U.S. Application Data
(60) Provisional application No. 62/003,065, filed on May 27, 2014.

(51) Int. Cl. B01D 71/28; B01D 71/70; B01D 61/02; B01D 61/14; C07C 51/47; C02F 1/44; C02F 1/40; C02F 1/66; C07C 29/76; B01D 69/12; B01D 61/58

(52) U.S. Cl. CPC B01D 71/28; B01D 69/125; B01D 71/70; B01D 61/022; B01D 61/145; B01D 61/58; C02F 1/442; C02F 1/444; C02F 1/40; C02F 1/66; C07C 29/76; C07C 51/47; B01D 2323/30; C02F 2001/007

(57) ABSTRACT

The present invention discloses stable composite membranes comprising a porous support having one or more thin selective layers coated on a top surface thereof, whereas at least one of said thin selective layers comprises a crosslinked fluorinated silicone polymer, and further wherein the total thickness of said one or more thin selective layers ranges between 0.1 to 10 microns. The use of these membranes in the process of olive oil wastewater treatment and the valorization of polyphenol-rich by-products, are also disclosed.

There is a significant problem in the management of olive oil waste streams, otherwise known as olive oil mill wastewater (OMWW or OMW). Olive oil waste streams cannot be discarded as they are ecologically toxic because of their chemical content. Furthermore, they cannot be used directly in agriculture as they are phytotoxic to fruits, vegetable and other plants in general.

On the other hand, these waste streams contain many valuable chemical components such as phenols and polyphenols.

Tyrosol and hydroxy-tyrosol are examples of phenols and polyphenols present in OMWW, each having a value of 80-200 Euro/kg, depending on the extent of its purification from the OMWW.

It would be of a significant commercial and ecological value if such valuable components could be cost effectively removed from olive oil waste streams thereby turning a waste into an important raw material for valuable compounds. To date there are no cost effective methods of separating the phenols and polyphenols from OMWW. In particular, the use of membrane separations has not shown to be effective, with currently available membranes lacking the necessary stability, permeability and/or polyphenol selectivity to be cost effective.

WO 2005/123603 (to Enea) discloses the selective fractionation and total recovery of polyphenols, water and organic substances from vegetation waters (VW), by a combination of acidification and an enzymatic hydrolysis followed by separation of the permeate streams obtained, by means of centrifugation and subsequent treatments with combined membrane technologies, using microfiltration (MF), ultrafiltration (UF), nanofiltration (NF) and finally a reverse osmosis (RO) membrane. Overall, at least 4 liquid/solid separation steps are needed to achieve the final product.

A later Enea publication (Russo, C., Journal of Membrane Science, volume 288, Issues 1-2, 1 Feb. 2007, Pages 239-246) discloses the same process, whereas the nanofiltration is optional, but it still has many separation steps, including the costly high-pressure RO step. Furthermore, the ENEA process is used for an OMWW containing a low amount of phenols and polyphenols (at most 350 ppm). Finally, the ENEA publications do not teach obtaining a concentrate enriched by high levels of phenols and polyphenols.

U.S. Pat. No. 8,066,881 discloses a method of obtaining a hydroxytyrosol-rich concentrate from olive tree residues by passing the waste flow stream through a nanofiltration membrane, followed by a feed compartment of a reverse osmosis unit, wherein the hydroxytyrosol and other bioactive compounds are retained and concentrated in a retentate stream. According to this process, the solid or semisolid residues and sub-products are preferably processed by extraction with biocompatible solvents prior to processing by nanofiltration. This process has numerous stages and involves solvents which can pose a serious waste problem. The fluxes with this process tend to be low, and the nano filtration of the biocompatible solvents requires more expensive solvent-stable membranes.

Livingston et al. (Journal of Membrane Science, 257 (2005) 120-133) teach a pilot scale application of a Membrane Aromatic Recovery System (MARS), composed of hollow fiber silicone membranes, for the removal of phenols and polyphenols from resin production condensates. However, these membranes are not sufficiently chemically stable to be cost effective at pH extremes needed to generate a polyphenols gradient across a membrane, and they have a relatively low flux/permeability of phenols.

The present inventors have now developed novel and improved composite membranes which can be useful for selectively removing polyphenols from olive oil wastewater streams.

As shown in the experimental section which follows, these membranes were formed by creating a highly selective thin layer or layers on a porous support, whereas this one or more selective layer is composed of at least one crosslinked fluorinated silicone polymer. The obtained composite membranes had superior selectivity towards a variety of polyphenols which exist in olive oil wastewater streams.

As is well known in the field of membranes and water treatment, polydimethylsiloxane (PDMS)-based membranes are not sufficiently stable under basic or acidic conditions.

However, in contrast, it has now been shown that the membranes' stability can be significantly improved over that of PDMS membranes by using fluorinated silicone polymers, in particular crosslinked fluorinated silicone polymers.

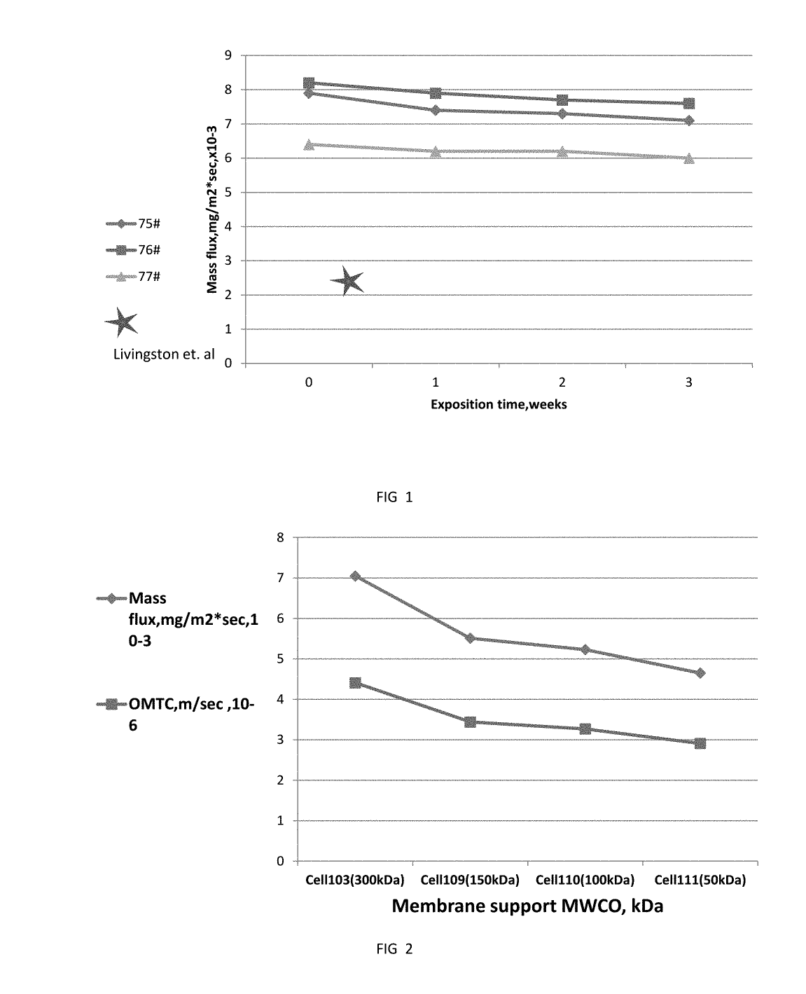

For example, as can be seen in FIG. 1, which shows the membrane phenol flux as function of exposure time at pH 13, the use of fluorinated silicone polymers such as Poly-trifluoro-propylmethylSiloxane (PTFS, membrane #75), as well as the mixing of PTFS with polyvinylphenol (PV4P, membranes # and 77) provided good stability under strong basic conditions.

It appears that the crosslinking of the fluorinated silicone polymers provides several advantages to the obtained membranes:
a) It imparts mechanical stability of the membrane;
b) It imparts chemical stability of the membrane so it doesn't get washed off during the process; and
c) It reduces the pore size to increase selectivity and prevent transfer of salts from the feed solution, and prevent transfer of base from the strip solution to the feed solution.

It has been further found that the thickness of the one or more selective membrane layers is an important aspect of their performance, and that in order to achieve the desired cost effective selectivity of the present membranes, a thin coating is required. It is expected that where the coating exceeds the desired thickness, the flux may be adversely affected.

In particular, it was found that the total thickness of the one or more thin selective layers should preferably range between 0.1 to 10 microns.

Thus, according to one aspect of the invention, there is provided a stable composite membrane comprising a porous support having one or more thin selective layers coated on a top surface thereof, whereas at least one of the thin selective layers comprises a crosslinked fluorinated silicone polymer, and further wherein the total thickness of this one or more thin selective layers ranges between 0.1 to 10 microns.

The term “membrane” as referred to herein may relate to a selective barrier that allows specific entities (such as molecules and/or ions) to pass through, while retaining the passage of others. The ability of a membrane to differentiate among entities (based on, for example, their size and/or charge and/or other characteristics) may be referred to as “selectivity”. More information regarding membranes may be found, for example, in http://www.bccresearch.com/membrane/DMDOO.html and http://www.geafiltration.com/glossary_filtration_terminologies.asp which are herein incorporated by reference in their entirety.

The term “composite membrane” as referred to herein may relate to a membrane that includes more than one material wherein the materials may have different densities. Composite membrane may include for example “thin film composite membranes” which may generally refer to membranes constructed in the form of a film from two or more layered materials.

However, it should be noted that although in one preferred embodiment of the present composite membrane is a thin film composite in flat sheet configuration, other embodiments of the invention include different configurations, such as, hollow fibers (HF) and tubular membranes. For hollow fibers and tubular membranes the selective layer can be within the lumen or on the exterior surface. Generally in flat sheet membranes the porous support (which is often an ultrafiltration membrane upon which the selective layer is coated) has a further underlying layer of a polymeric nonwoven layer often made from polyesters or polyolefin. In case of HF or for relative small diameter (<5 mm by way of a non-limiting example) tubular membranes a nonwoven support layer may not be necessary since the base membrane material forming the hollow fiber wall has inherent mechanical strength.

The term “porous support”, also referred to as a “porous membrane”, or a “support layer”, and refers to the layer that provides a mechanical support for the selective layer. The support layers are non-selective, and not considered the selective part of the membrane. In the state of art the support layer of a composite is considered part of the membrane.

Examples of porous supports suitable for the present invention, include, but are not limited to, ultrafiltration membranes, microfiltration membranes and nonwoven polymers such as polysulfone, polyethersulfone, polypropylene, or polyvinylidene difluoride (PVDF).

The effect of the nature of the support appears to be significant in controlling mass transfer wherein the use of a UF support gave much higher permeability and mass transfer rates of phenol (see for example membrane #98 in Table 1) compared to when the membrane was made on a polypropylene (PP) nonwoven support (membrane #100 in Table 1). Furthermore, the intrinsic permeability of the UF 50 kDa support was much higher than that of PP nonwoven support (see last two entries in Table 1).

Thus, according to one preferred embodiment of the present invention, the porous support is a UF membrane. The UF membrane may or may not have an underlying additional nonwoven support, but in this case the selective layer is on the upper surface of the UF membrane.

The materials of the UF support should be stable to the pH extremes that will be used on the feed and permeate side and should be stable to any solvent effects of the components of the olive oil waste streams.

Some preferred materials for the UF membranes are engineering plastics such as polysulfone, polyethersulfone, polyphenylsulfone, polyether ketone, polyether-ether ketone and their combinations. Crosslinked and solvent- and pH-stable UF membranes may also be used. The UF membrane may also be further supported by non-woven supports as for non limiting examples from polypropylene, other polyolefins and polyesters.

In one example, the ultrafiltration membrane is a polyethersulfone polymer. In another embodiment the UF membrane used as a support has a MW cutoff (MWCO) of 50 kDa, 100 KDa, 150 Kda or 300 kDa.

As shown in Example 2 and Table 2, by using UF membranes of 150K and 300K pore sizes the flux and permeability of the composite invented membrane increases.

The term “selective layer”, as used herein, refers to the actual membrane which mediates the permeation of all species through the membrane, imparting the greatest flow resistance and deciding the selectivity of the composite membrane. Usually, it has the narrowest pore structure or smallest domains of free volume (if the selective layer is a dense non-porous layer) and specific chemical structure, which together define what chemical species are capable of passing through it.

The term “thin” with regard to the term “thin membrane” refers to a total thickness of the one or more thin selective layers ranging between 0.1 to 10 microns, more preferably between 1 to 5 microns.

This means that if there is a single layer, its thickness can be up to 10 microns, preferably up to 5 microns, but that if there are more layers, it is their added thickness that cannot exceed 10 microns. For example, if there are four layers, their added thickness cannot exceed 10 microns, thus, in one example each layer can have a thickness of up to 2.5 microns, or some can have a lower or higher thickness, as long as the added, or total, thickness does not exceed about 10 microns.

It is important to note that the coating is conducted only on the upper selective side of the support membrane, and that no coating is applied to the under, more porous, side of the support membrane.

As noted hereinabove, the selective layer described herein comprises at least one crosslinked fluorinated silicone polymer.

The fluorinated silicone polymer is selected from, but not limited to, fluorinated polysiloxanes, fluorinated polysilanes, fluorinated chlorosilanes, fluorinated alkoxysilanes, fluorinated aminosilanes, fluorinated silicone esters, fluorinated polydialkylsiloxanes, and phenyl substituted fluorinated polysiloxanes.

Preferably, in the composite membrane described herein the fluorinated silicone polymer is a fluorinated polysiloxane.

One source of fluorinated silicones is Siltech which offers a series of fluorinated silicones as well as fluorinated silicones that also contain alkyl or polyether pendent groups.

In one preferred embodiment of the present invention, the fluorinated polysiloxane is Poly-tri-fluoro-propylmethyl-Siloxane.

In another preferred embodiment, for some advanced applications, fluorinated silicones carrying phenyl groups may be used.

Commercially available polysiloxanes have molecular weights between 1000 and 300,000 gr/mol, although the invention is not limited to this range.

The amount of the fluorinated silicone polymer should range from 20% to 100% in the final film composition after the solvent evaporates and the film is cured. This corresponds to a concentration of 0.1-10% in the coating solution.

The term “crosslinked” or “crosslinked polymer” as used herein means that the polymer chains of the fluorinated silicone polymers are bonded to one another.

While having at least one crosslinked fluorinated silicone polymer is a necessary part of the invention, according to another preferred embodiment the selective layer(s) further comprise a non-crosslinked fluorinated silicone polymer, which can be any of the fluorinated silicone polymers listed above. Using the non-crosslinked fluorinated silicone polymer is advantageous in that the mixtures are able to control pore structure size and chemistry and contribute to membrane selectivity and permeability.

The amount of the non-crosslinked fluorinated silicone polymer should range from 5% to 20% of the coating weight.

According to yet another preferred embodiment, the one or more thin selective layers further comprises a non-fluorinated silicone polymer.

Examples of non-fluorinated silicone polymers include, but are not limited to, dimethyl polysiloxane, methylphenyl polysiloxane, silicone esters, polysiloxanes, polysilanes, chlorosilanes, alkoxysilanes, aminosilanes, polysilanes, polydialkylsiloxanes, and phenyl substituted polysiloxanes.

In one preferred embodiment, a non-fluorinated silicone polymer serves as a pore protector of the support layer.

The term “pore protector” refers to a compound, often a polysiloxane, which is used in absence of curing agents or catalysts), serves the dual purpose of preventing the pores from collapsing, when the support is dried during the curing of the silicone layer, and of preventing passage of the coating material deeply into the pores and thus also preventing an undue reduction of the flux of the finished coated membrane.

It has now been further found that the invented membranes have achieved overall phenol permeability and in some embodiments selectivity for given components of OMWW by incorporating additives with selective uptake of phenols, into the silicone membranes.

For example, it has been found that the selectivity of the composite membranes of the present invention towards phenols and polyphenols found in the olive oil wastewater, can be increased by the addition of at least one polyphenol and/or at least one polymer having one or more aromatic hydroxyl groups per monomer and/or at least one monophenol, to the one or more selective thin membranes.

In one example, as seen in Table 1, the phenol mass transfer rate was somewhat higher for PTFS-polyphenol (PV4P) membranes as compared to only PTFS membranes. For example, membrane #97 (PTFS/PV4P blend) showed a 25% increase in the overall mass transfer coefficient (OMTC, in m/sec) over membrane #75 (PTFS only). In another example, the silicone tubing in FIG. 1 (Dana/Livingston) gave a mass flux of only 2.3*10⁻³ mg/m²-s compared to 3.8 mg/m²-s in membrane #98 in Table 1 (PTFS/PV4P).

The effect on selectivity and mass transport rates for the membranes of PTFS with added PV4P are shown in Tables 3 and 4. PV4P membrane showed higher selectivity for tyrosol and hydroxtyrosol. For example, in membrane #77 (PTFS:PV4P=6:4) the selectivity was 4.0, as compared to 3.8 in a membrane of only PTFS (#75).

As further shown in the experimental section which follows after 30 hours of operation the tyrosol/coumaric selectivity factor (Cp/Cf)tyrosol/(Cp/Cf)coumaric increased from 1.15 for a pure PTFS membrane (#75) to 2.4 for a PTFS:PV4P membrane (#97).

According to one preferred embodiment, the polyphenol may be a relatively low molecular weight (such as hydroxyl-Tyrosol) or a polymer (such as a poly vinyl phenol, PV4P) and copolymers comprising one category of monomers with phenols or polyphenol.

The term polyphenol as used herein includes both polymers and oligomers containing multiple phenolic units, in particular those found in OMWW. However, the term “polyphenol” is sometimes used in a broader sense and also encompasses any compound that has more than one aromatic hydroxyl group, such as tyrosol and/or hydroxytyrosol and/or resorcinol.

Wherever it is intended to refer specifically to a low-molecular weight phenol monomer, the term “phenol” or “monophenol” is also used.

Preferably, the polyphenol is a poly vinyl phenol.

Various ratios of a fluorinated polymer and a polyphenol have been tested, and while a broad range can be selected, ranging from a ratio of fluorinated polysiloxane polymers and oligomers:polyphenol=9:1 to a ratio of fluorinated polysiloxane polymers and oligomers:polyphenol=1:1.

In one preferred embodiment, shown in Tables 3 and 4, it can be seen that the preferred ratio of PTFS:PV4P is 6:4 (wt:wt).

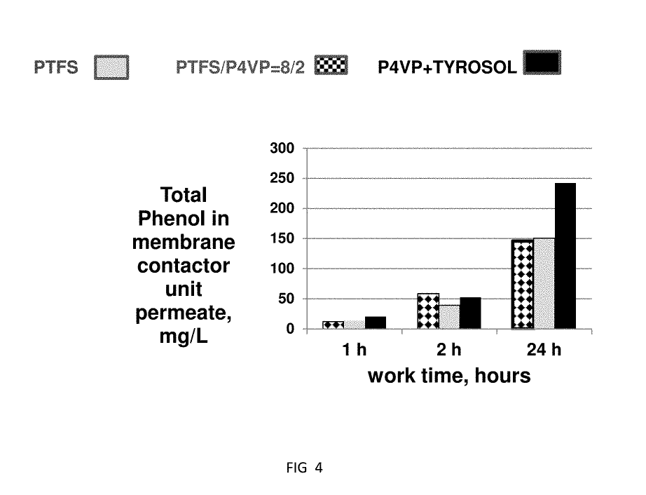

As seen in FIG. 4, the total phenol permeability can be significantly enhanced by the addition of rational additives that form channels for phenol passage. For example, while the permeability of a selective layer of only P4VP is very low with little total phenol passage under the above conditions, the addition of tyrosol to the selective layer significantly enhanced phenol permeability.

Thus, according to yet another preferred embodiment, the polyphenol additive can also be a low molecular molecule, such as but not limited to, tyrosol, phenol, resorcinol, hydroxy-benzoic acid.

Other low molecular weight phenols such as phenols having different alkyl groups such as methyl, ethyl or propyl groups in the ortho, meta or para positions on the phenyl group may be used by way of non-limiting examples.

Tyrosol is a monophenol found in OMWW. Therefore, according to a preferred embodiment of the present invention, the monophenol is tyrosol.

The low-molecular weight monophenol can be added either alone, or in addition to an oligomeric or polymeric polyphenol.

The polyphenols and/or monophenols may or may not be covalently bound to the polymers of the selective layers.

In particular, if the low molecular weight non polymeric phenols are not covalently bound to the polymers of the selective layer, they can consequently leach out from the selective layer and leave fixed, well defined spaces, that increase membrane selectivity.

Alternatively, according to another preferred embodiment of the present invention, the composite membrane described herein may comprise a polymer having one or more aromatic hydroxyl groups, whereas this polymer is derived from plastics selected from: hydroxylated polysulfone, polyethersulfone, polyphenylene oxide, polyetherketones, aromatic polyamides, and hydroxylated engineering plastics polymerizted by condensation polymerization and copolymers of hydroxylated polystyrenes prepared by chain reaction polymerization.

The composite membranes of the present invention may comprise one or more thin selective layers, so long as the total thickness thereof is thin enough to permit sufficient flux, namely a total thickness of up to about 10 microns.

In one preferred embodiment of the invention, the composite membrane comprises a single thin selective layer. This thin layer comprises at least one crosslinked fluorinated silicone polymer, and optionally comprises additional components, such as, but not limited to, a non-crosslinked fluorinated silicone polymer, a non-fluorinated silicone polymer, a polyphenol and a monophenol.

As can be seen from the results of Table 1, one preferred composition of a single layer thin selective membrane is a combination of a crosslinked fluorinated silicone polymer and a polyphenol, such as the combination of Poly-trifluoropropylmethylSiloxane (PTFS) and polyvinyl phenol (PV4P).

In another preferred embodiment of a single layer of the selective membrane, the thin selective membrane further comprises Tyrosol, which enhances the permeability and selectivity of the monophenols present in the OMWW.

As demonstrated in Example 3, multilayer membranes are of enhanced performance over single layered membranes.

In one particular preferred embodiment is a composite bilayer membrane, namely a composite membrane wherein the thin selective membrane comprises two selective layers.

According to one preferred embodiment, the first of these two layers comprises crosslinked Poly-trifluoropropylmethylSiloxane (PTFS) and polyvinyl phenol, and a second of these layers comprises polyvinyl phenol and tyrosol.

In one particular preferred embodiment there is provided a composite membrane comprising three selective layers.

According to one preferred embodiment, both a first and a last of said layers comprises a crosslinked Poly-trifluoropropylmethylSiloxane (PTFS), and a second of said layers, in between the first and the last layers, comprises polyvinyl phenol and tyrosol.

By the term “first layer” it is referred to the layer which is first coated on the porous support, and the terms “second layer”, “third layer” etc. refer to the layers which are coated on top of the first layer, such that the “last layer” to be coated would become in fact the top layer of the composite membrane.

As can be seen in the Examples section which follows, the present invention successfully teaches the preparation of the novel composite membranes taught hereinabove.

As noted above, in one important aspect of the present invention, the selective layer comprises crosslinked fluorinated silicon polymers. Thus, in the preparation of the composite membrane of the present invention, it is important to include in the coating solution at least one fluorinated silicon polymer which can be crosslinked, namely at least one fluorinated silicon polymer that has a crosslinkable group.

The term “crosslinkable group” as used herein means a group capable of crosslinking the silicone polymer compound. The crosslinkable group is not particularly limited as long as it has such a function, and it is preferably a functional group capable of undergoing either an addition polymerization reaction or a functional group capable of generating a radical by irradiation.

One preferred example of a crosslinkable groups of fluorinated silicone polymers are terminal hydroxyl groups, noted as Si—OH groups.

The concentration of silicone in the coating solution may vary from 0.01 to 10%, but is preferably in the range of 0.1 to 2%, for both the initial coating step and the final coating step.

The fluorinated and non-fluorinated silicones of the present invention are crosslinked in the presence of a crosslinking agent.

The term “crosslinking agent” or “crosslinker”, as used interchangeably in the present application, refers to any compound that can chemically react to link two other compounds together. The chemical reaction can include hydrosilylation.

The crosslinking agent may be selected from several chemical groups:

One preferred group of suitable crosslinking agents are organic and inorganic peroxides. These crosslinking agents are typically used when olefinic bonds are present in the silicones.

Organic peroxides include, for example, dicurnyl peroxide, 2,5-dimethyl 2,5 (ditertiary butyl peroxy) hexane, di-tertiary butyl perphthalate, tertiary butyl hydroperoxide, and others.

Suitable choice of type and quantity of organic peroxide used as a cross-linking agent should be made dependent upon the type of polyolefin resin and desired degree of cross-linking, but it is preferable that less than 3% by weight of peroxide compound based upon weight of the resin, be used.

Moreover, cross-linking reaction of polyolefin may be performed by using polyfunctional monomer, such as divinyl benzene, etc, or acetylene. Such monomers are used with cross-linking agent, as agents which increase efficency of cross-linking polyolefin.

Another preferred group of suitable crosslinking agents are poly alkyl siloxanes containing silanic hydrogen. This is useful for the crosslinking of silanol-terminated silicone polymers.

Yet another preferred group of suitable crosslinking agents are poly alkoxy silanes. This is most suitable for the crosslinking of silanol-containing or -terminated siloxane polymers. Some examples of an alkoxysilane include, but are not limited to, tetraalkoxysilane, trialkoxysilane or polyalkoxysiloxane.

One specific preferred example of a tetraalkoxysilane crosslinking agent is tetraethoxysilane.

According to preferred embodiments of the invention, the crosslinking agent is an alkoxysilane selected from tetraethyl orthosilicate (TEOS) or fluorinated tetraethyl orthosilicate.

The term “silane” as used herein refers to any compound having the formula Si(R)₄, wherein R is independently selected from any hydrogen, halogen, or optionally substituted organic group; in some embodiments, the organic group can include an organosubstituted siloxane group, such as an organomonosiloxane group, while in other embodiments, the organic group does not include a siloxane group.

The concentration of the crosslinking agent may vary between 0.05 and 10%, preferably 0.1 and 5%.

Increasing crosslinking ultimately reduced mass flux (MF) and OMTC (see for example sample #93 having 0.5 ml crosslinker vs. sample #95 with 0.8 ml cross-linker). At the same time, by increasing the PV4P in the blend (see for example, membrane #97), cross-linking at a value intermediate (0.6 ml) between membrane #93 and #95 gave higher OMTC than both. In this case, the effect of adding PV4P was stronger than the effect of increasing cross-linker concentration in increasing OMTC.

Table 5 shows the effect of selective membrane crosslinker concentration on composite Membranes (for a 50K UF support) in terms of phenol flux and Salt rejection (1000 ppm NaCl). The results show that increasing the concentration of the crosslinker TEOS, increases NaCl rejection without significantly hurting phenol permeability. The high salt rejections are needed for maintaining the pH gradient across the membrane in the membrane contactor, so that sodium hydroxide does not diffuse from the strip side to the feed side. The comparative results (carried out with silicone tubing) had significantly lower (3 to 4 times) OMTC.

Preferably, in addition to the crosslinking agent, a catalyst is also added. By a suitable choice of catalyst, the curing may be effected at room temperature at a time ranging from 30 minutes to 4000 minutes.

The term “catalyst”, as used herein, refers to compounds which are capable of increasing the polymerization rate of a polymer-forming material, in this case the crosslinking of silicone polymers.

Presently preferred catalysts are stannous octoate, and dibutyltin dilaurate. Other possible catalysts are dibutyltin dioctanoate, dibutyltin diacetate, salts of carboxylic acids such as iron 2-ethylhexanoate and cobalt naphthenate, titanic acid esters, and amines such as ethylamine, dibutylamine and pyridine.

In one preferred embodiment, the catalyst is selected from stannous octoate and dibutyltin dilaurate.

The catalyst is preferably added in an amount ranging from 0.1 to 2% of the coating solution.

The term “solvent” will be well understood by the average skilled reader and includes an organic or aqueous liquids with molecular weight less than 300 Daltons. It is understood that the term solvent also includes a mixture of solvents.

Solvents suitable for the present invention are selected from aliphatic solvents and/or from perfluoro solvents.

The term “aliphatic solvent” includes aliphatic or alicyclic hydrocarbon solvents which may be linear or branched and/or optionally substituted, such as for example pentane, hexane, cyclohexane, heptane, octane, isooctane, methyl cyclohexane or dekalin or mixtures thereof.

One preferred example of an aliphatic solvent is hexane.

As used herein, the term “perfluoro solvent” refers in fact to solvents which dissolve perfluorinated materials.

The term “perfluorinated materials” as used herein refers to fluorinated silicone polymers.

One preferred example of such a suitable solvent is Tetrahydrofuran (THF).

To summarize so far, the first step in the preparation of the composite membranes of the present invention, is the preparation of a coating solution comprising a crosslinkable fluorinated silicone polymer, a crosslinking agent, a catalyst and a solvent, and optionally a polyphenol and/or a monophenol.

One preferred combination is silanol-terminated siloxane (MW 36,000) with tetraethoxysilane as crosslinking agent, and dibutyltin dilaurate as catalyst, coated onto a porous substrate such as an ultrafiltration membrane, from an aliphatic hydrocarbon solvent such as hexane, or from perfluoro solvents, such as THF.

The solvents for the pore protector added to the pores of the UF support prior to coating the selective layer are e.g., lower (e.g. C₁ to C₄) alcohols; or the same solvent could be used for both the pore protecting step and for the final coating step. The pretreatment with the pore protector may be carried out, for example, by dipping the membrane into a dilute solution of the pore protector in a low-boiling inert solvent, e.g. a low boiling alcohol having 1-4 carbon atoms, such as methanol, ethanol, propanol or butanol. The final silicone coating and the pore-protecting silicone layer should desirably have a thickness in the range of from 500 to 5000 Å, more preferably in the range from 1000 to 2000 Å.

Once the coating solution has been prepared, the silicone polymer may be applied onto the support membrane.

Although the coating can be sone on many kinds of substrate membranes, in a preferred embodiment the selective layer is coated onto an “ultrafiltration” or “UF” membrane wherein the molecular weight cut off (MWCO) of the support membrane may vary from 5K to 500K.

In one preferred embodiment, the membranes are prepared by phase inversion methods to form an integrally skinned top layer which defines the MWCO. The membrane may be flat sheet, tubular, and hollow fiber. Each can be used within the present invention. In one preferred embodiment the UF supporting membrane has a MWCO of between 20K to 300K.

The support membrane may or may not be a pore-protected support, whereas pore-protection may be conducted in many different ways known in the art of coating thin films onto porous supports. Such methods are described, for example, in U.S. Pat. Nos. 4,243,701, 4,230,463, and 4,950,314 and in J. Membr. Sci., 1976, 1:99.

One presently relatively simple method is dipping two sheets of the porous substrate which are temporarily glued together back to back (with their tight small pore side facing outwards) into a solution of silicone polymer or prepolymer, draining and curing.

After dipping, the coated support is drained at room temperature and left to stand under controlled conditions such as in a hood for different periods of time, as for one example for 72 hours at room temperature in the hood. Usually at room temperature, the curing time ranges from 30 minutes to 4000 minutes. Shorter time of curing can be done at elevated temperatures such as 50° C. for 1 to 2 hours, but can be done at higher temperatures, for example at about 85° C. There is a time-temperature optimization that is easily determined by standard optimization.

As noted hereinabove, the overall thickness of the selective layers ranges between 0.1 to 10 microns, more preferably between 1 to 5 microns.

The determination of the thickness of the final layer is done by calculation based on the concentration of the polymer in the coating solution and further by controlling the solution thickness as it is applied to the membrane by mechanical means.

Preferably, the amount of polymer in the solution, that is needed to achieve the final required thickness of 0.1 to 10 microns, after curing and evaporation of the solvent, ranges from between 0.1% to 10%, more preferably between 0.5% to 10% by weight.

In one embodiment, the coating can be done using a blade or a knife and then the wet film thickness and final dry film thickness is easily achieved by adjusting blade and knife position above the substrate to be coated by well known state of art methods and calculations.

For multilayer membrane preparation, after each layer the coated support is drained in hood for about 30 minutes and after this is cured in oven for 1 hour at about 50° C. After curing the support is cooled for about 15 minutes at room temperature.

As noted hereinabove, several layers may be prepared and coated, as long as the total thickness is as described.

In one embodiment the first layer may be a phenol interacting layer such as a polyphenol (for example PV4P), with optional additional low molecular weight components added to enhance polyphenol permeability (such as tyrosol or other mono or polyphenols such as hydroxyl tyrosol).

In another embodiment the low molecular weight components, such as the tyrosol may be leached out, if they are not crosslinked, and upon leaching may confer additional permeability and or selectivity to the membrane.

Thus, according to another aspect of the invention, there is now provided a process for the preparation of the composite membrane described hereinabove, this process comprising:

a) Preparing a first coating solution comprising a crosslinkable fluorinated silicone polymer, a crosslinking agent, a catalyst and a solvent, and optionally a polyphenol and/or a monophenol;

b. contacting the coating solution onto a top surface of a porous support thereby forming a layer onto said support;

c. curing said layer for a time ranging from 30 minutes to 3000 minutes at a temperature ranging from 20° C. to 85° C., to obtain a first stable thin selective layer having a thickness ranging between 0.1 to 10 microns;

d. optionally further preparing one or more additional coating solutions, each comprising one or more of a crosslinkable fluorinated silicone polymer, a fluorinated silicone polymer, a non-fluorinated silicone polymer, a crosslinking agent, a catalyst, a polyphenol, a monophenol, and a solvent; contacting said one or more additional coating solutions with said first thin selective layer, and curing said additional layers, so as to obtain a total thickness of said one or more thin selective layers ranging between 0.1 to 10 microns.

The term “contacting” in relation of the coating solution is intended to include any type of contacting, examples of which include, but are not limited to, coating, blending, dipping, and the like, and other methods known to the art.

The term “stable” with regard to the thin selective layer of the present invention includes both chemical stability as well as stability under acidic or basic conditions.

As noted in the background section hereinabove, state of art membrane aromatic recovery system (MARS) technology is characterized by a low flux or permeability of phenols and polyphenols across the membrane. Another disadvantage in presently-known MARS technology is the limited chemical resistance of its membrane to the extreme pHs needed for improving the flux/permeability rates of the membrane.

In contrast, the composite membranes of the present invention have an improved phenol and polyphenol flux/permeability, and are highly stable under acidic and basic pH.

Furthermore, the composite membranes of the present invention have a high selectivity towards the phenols and polyphenols found in olive oil waste streams, in particular for the recovery of tyrosol and hydroxytyrosol.

Table 7 shows that the results from using a two layer membrane (#80) were somewhat better than that of the single layer membrane (#77), both in terms of passage of hydroxytyrosol and tryosol, and in terms of selectivity (ratio showing hydroxytyrosol and tryosol as fraction of the total organic carbon in the sample) which is higher from membrane #80. This demonstrates a significant enrichment of using multiple layers and especially for a double layer. It also shows the importance of including a low molecular weight phenol, such as tyrosol, into at least one of the layers.

The above table shows that of the two membranes tested in parallel on the given feed, Membrane 80 was clearly superior. It had both high permeability to phenol (0.537 g/L permeated in one day), and high selectivity (polyphenol was 25% of all TOC in the permeate up from 10% in the feed).

It should be noted that this preferred membrane had three components (PTFS, PV4P and Tyrosol). It is believed that the addition of tyrosol, which eventually leached out, helps to form a crosslinked structure of PTFS/PV4P with high permeability and selective passage of polyphenols.

Therefore, according to another aspect of the invention, there is now provided an improved system for the valorization of olive oil waste streams, this system comprising of a novel membrane contactor unit based on the composite membranes of the present invention.

The term “system” as used herein refers to an interconnected assembly of components, in this case a membrane contactor unit.

The term “stream”, as used herein, is interchangeable with the term “flow”, and refers to a moving or still form in a container, vessel, or processing equipment.

In the context of the present invention, the term “wastewater stream” is intended to mean an aqueous solution containing water, organic compounds and one or more further organic or inorganic component deriving from olive oil production processes, such as olive oil milling. The olive oil wastewater stream is otherwise known as OME or OMWW.

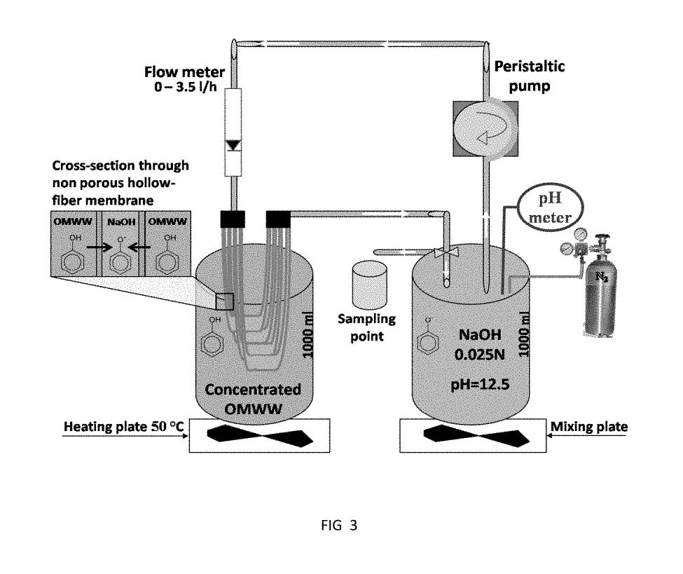

FIG. 3 is a scheme showing the membrane based extraction of phenolic compounds from OMWW using the membrane contactor unit comprising the composite membranes of the present invention. The pH difference between the feed (pH ˜2) side and the permeate (pH ˜13) side was used to establish a gradient to polyphenol by the formation of polyphenoxide on the permeate side.

In particular, acidic pH was used on the feed side to ensure that all the phenols are protonated and not charged. Basic pH was used on the permeate side to form anionic phenolates which therefore do not penetrate the membrane and establish a zero concentration of uncharged phenols, thereby maximizing the concentration gradient of phenols across the membrane and creating a powerful driving force.

The permeate comprising the phenolates can be returned to the membrane contactor unit for yet another cycle of extraction. Thus, with time, the % of the phenolate in the permeate continuously increases as more and more solids pass the contactor membrane.

Thus, according to another aspect of the present invention, there is provided a membrane contactor unit, comprising the composite membrane of the present invention, in which the selective side of the membrane faces a feed stream rich in polyphenols whereas the porous side of the membrane is adjacent to a high pH strip solution.

As used herein, the term “strip solution” is used interchangeably with the term “stripping solution” and refers to an aqueous solution that mediates back extraction of an ion from an organic containing solution. Examples of high pH strip solution include but are not limited to NaOH, KHCO₃, NaHCO₃, K₂CO₃, or Na₂CO₃. Preferably it is a NaOH solution.

The term “membrane contactor” or “membrane contactor unit” is used to identify membrane systems that are employed to keep in contact two phases under flowing conditions.

Using this membrane contactor unit creates a cost-effective separation, concentration and valorization process to obtain OMWW by-products.

The term “valorization” as used herein is intended to mean the usage of the by-products of the OMWW as a raw material of a value added product.

The term “by-product”, as used herein interchangeably used with the terms “product”, “concentrate product”, “polyphenol-rich product” or “polyphenol product” refers to by-products of olive of extraction, and includes in particular polyphenols and phenols found in OMWW.

This process is advantageous in many aspects. For one, no costly high-pressure RO stage or centrifugation are needed (as in the prior art processes), and the number of separation stages is much lower (2 stages vs. 3-4 in the prior art). Furthermore, in prior-art processes undesirable salt concentrates are obtained and need to be handled, whereas in the presently developed system salt concentrates are avoided altogether, since the permeate can be run through a biological treatment. Yet another advantage of the present system is that the UF or NF membrane of the present invention is not a ceramic membrane but a polymeric membrane which is considerably less expensive and not as brittle and fragile as ceramic membranes.

Another important advantage in the present process is that minimal amount of base are required to maintain a polyphenol gradient, since the phenolate cannot pass back through the membrane.

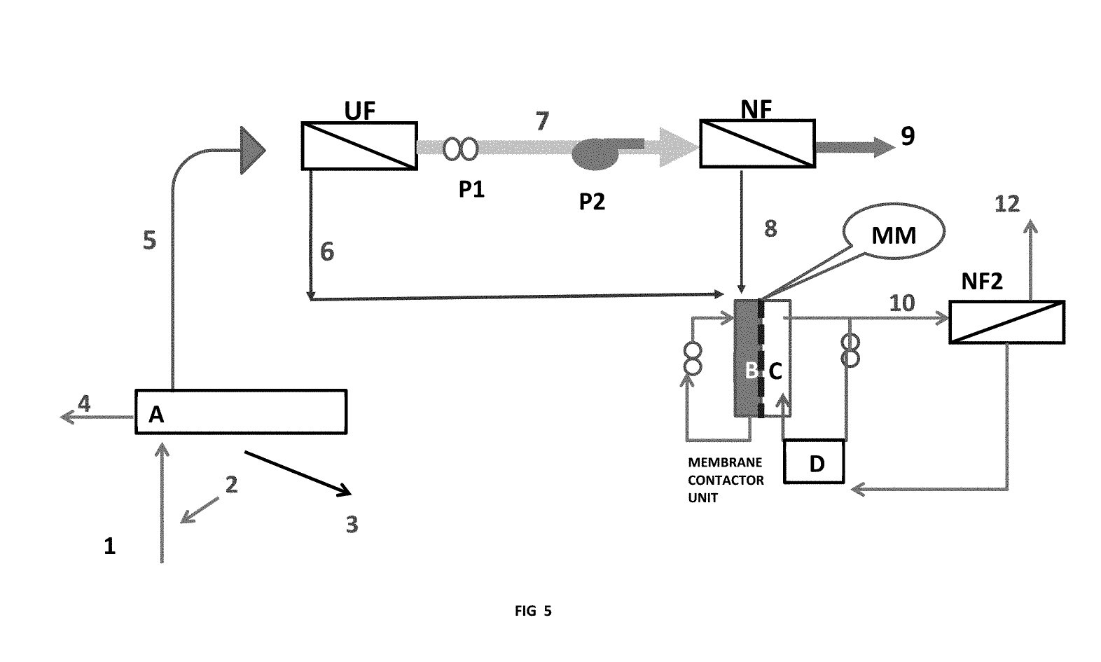

As can be seen in FIG. 5, in a typical olive oil wastewater (OMWW) treatment process according to the present invention, the olive oil mill wastewater stream (1) is fed to a holding tank (A) along with acid (2) which is added to acidify the olive oil mill wastewater to pH=2-2.5 in order to stabilize and prevent fermentation, as well as hydrolyze Oliopeurine to cleave the hydroxytyrosol from its glycosidic moieties.

In tank A there are phase separations of a bottom layer (3) rich in suspended solids and of a top layer (4) rich in olive oil. In particular, the solid rich layer (3) is drained from the bottom to remove the suspended solids from the OMW, and the oil rich layer (4) is mechanically skimmed or decanted from the top, to recover the oil. The recovered oil is used as part of the produced oil and can be marketed for uses appropriate to its quality. The suspended solids can be used for composting or to generate biogas.

The remaining OMW (5) is fed to an ultrafiltration module or modules unit (UF) which removes all suspended solids and some of the polyphenols as well. A pump (P1) is used to pull the permeate through the UF membrane if the membrane is submerged in the treated OMW feed (5) or else a pressurized pump is used upstream of the UF unit and pushes the permeate through the membrane. The concentrate from the UF unit (6) is fed into the left side of the membrane contactor unit (B) to recover the polyphenols and is recycled next to the selective layer of the membrane. The permeate of the UF unit (7) is fed to a pump (P2) which then pressurizes the UF permeate and sends it to nanofiltration membrane module or modules (NF) where most of the polyphenols are retained in the concentrate (8) thereby obtaining a permeate (9) which is largely free of polyphenols (80-90% reduction). This permeate can be sent to municipal wastewater treatment plant for standard biological treatment. The NF concentrate (8) is fed to the left side of the membrane contactor unit (B) where it is recycled next to the selective membrane (MM). A high pH strip solution (D), at a pH ranging from 11 to 13, is recycled next to the porous side of the membrane contactor unit (C). When it is sufficiently loaded with polyphenol it can be abstracted as a loaded strip stream (10) for subsequent concentration of the permeated polyphenols using another nanofiltration unit (NF2) which will have very high retention of the polyphenols because the they are charged at high pH. The caustic solution that permeates the NF2 unit (11) can then be recycled to the membrane contactor unit to strip out more polyphenol from streams 6 and 8. The polyphenol concentrate from the NF2 unit (12) is either of a concentration that it can be used directly, or it can be neutralized and further purified on a chromatographic column to reach over 98% purities. Typically, after NF2, the phenols can be at least 5% and preferably above 10% w/w. This is a substantial increase of the amount of phenols in the actual solution to about 50-100 g/L whereas the contactor permeate comes out only at about 0.5-5 g/L.

In general, using this process resulted in membranes showing a significant increase in the total phenol concentrations in the final permeate, as compared to the feed. For example, typical total phenol content in the raw olive oil waste may be about 0.2-1% of the TOC, and increases to about 4%-10% of the TOC in the concentrate after the first nanofiltration (NF). After passage though the membrane contactor unit of the present invention, as detailed in the sequence of steps disclosed hereinabove, the total phenol content of the permeate increases to 20-25% of the TOC.

Thus, another aspect of the present invention is a process for obtaining a polyphenol rich concentrate of an olive oil mill wastewater stream, this process comprising:

a. Contacting an olive oil mill wastewater stream with an acid, to obtain an acidified olive oil mill wastewater stream at a pH ranging from 2 to 2.5;
b. Feeding the acidified olive oil mill wastewater stream into a holding tank, and phase separating from the olive oil mill wastewater stream:
i. a bottom layer stream rich in suspended solids,
ii. a top layer stream rich in olive oil,
iii. a middle layer stream, that is largely devoid of suspended solids and of olive oil;

c. Feeding the middle layer stream into an ultrafiltration unit (UF), thereby separating the middle layer stream into a UF permeate and a UF concentrate;

d. Feeding the UF permeate into a nanofiltration unit (NF), thereby separating the UF permeate into a NF concentrate rich in polyphenols and a NF permeate largely free of polyphenols;

e. Separately feeding each of the UF concentrate and the NF concentrate into a selective side of a membrane contactor unit of the present invention, and circulating the concentrate next to the selective side of the membrane, further whereas a high pH strip solution is circulated next to a porous side of the membrane, to obtain a polyphenol rich permeate stream at the porous side of the contactor membrane unit; and

f. passing the polyphenol rich permeate stream through a second nanofiltration unit (NF2), thereby obtaining a concentrate which is a polyphenol rich product, and a caustic solution permeate.

As used herein, the term “permeate” refers to the stream passing through the membrane surface, while the term “concentrate” defines the portion of the stream exiting the filter or membrane, containing retained, non-permeating species.

It should be noted that the term “holding tank” generally refers to any vessel or conduit in the wastewater stream at which the wastewater may be held and/or separated and is not limited to any particular type or structure of tank or vessel.

Further it should be noted that the present system and process is appropriate for use in a flow-through process, in which wastewater continually flows into and out of the holding tank, or in a batch process, in which holding tank is filled, treated and then emptied.

The term “largely devoid”, with regard to the middle stream, refers to a composition having less than 5% of either suspended solids and/or of olive oil. More preferably, less than 1% of suspended solids and/or less than 3% of olive oil.

According to a preferred embodiment of this process, the polyphenol rich concentrate product comprises at least 5 w/w % phenols and/or polyphenols, more preferably at least 10 w/w % phenols and/or polyphenols.

According to a preferred embodiment of this process, it further comprises purifying the polyphenol rich concentrate from NF2.

Regarding the biodegradability of the NF permeate, In a batch experiment, a diluted sample of NF permeate (1:5) which contained ˜600 mg/L of dissolved organic carbon was degraded to 20 mg/L of dissolved organic carbon over 144 hours (see Example 4 below).

Thus, according to a preferred embodiment of this process, it further comprises passing the first NF permeate through a biological treatment unit, to obtain an irrigation-adequate stream having a chemical oxygen demand (COD) lower than 300 mg/L.

The term “chemical oxygen demand”, abbreviated COD, is used in its usual sense of denoting the total oxidizable material present in the liquid under consideration regardless of whether or not it is biodegradable. The term “BOD” denotes the amount of oxygen consumed during a 5-day period of bacterial activity on a chemically standardized and stabilized sample. While COD is not strictly comparable to the Biological Oxygen Demand (BOD) it is useful as an indication of reduction of BOD to give a basis for comparison of the effectiveness of alternate methods of treatment, particularly when applied to comparable waste samples.

According to a preferred embodiment of this process, it further comprises recycling the caustic solution permeate into the porous side of the membrane contactor unit, thereby stripping out additional polyphenol.

The invented membranes may be used to recover polyphenols from many different sources and in one important embodiment it can be used for extracting phenols and polyphenols, and in one embodiment for enhanced recovery of hydroxytyrosol, and in yet another embodiment for the recovery of tyrosol, from olive oil wastewater stream generated in olive oil production.

Thus, yet another aspect of the present invention is a use of the composite membrane of the present invention in obtaining a polyphenol rich concentrate product of an olive oil mill wastewater stream.

The invented membranes may be used on the original olive oil wastewater stream, or it may be used on olive oil wastewater stream that has been pre-treated with ultrafiltration (UF), and/or Nanofiltration (NF) and or reverse osmosis (RO). In the case of samples pretreated with NF or RO or UF followed by NF or RO, the concentrate of the NF or RO is treated with the membrane contactor units of the present invention.

The invented membranes may be used in both dialysis cells and a flow cell similar to a Membrane Aromatic Recovery System (MARS). The invented membranes may be in flat sheet configuration, hollow fibers or tubular configurations. In flat sheet configurations the membranes may be in plate and frame systems or in spiral wound configurations both well known in the state of art.

In tests with olive oil waste stream concentrates taken from NF or RO pre-treatments, and run through a membrane contactor unit in a flow cell for the membrane #75 (PTFS), it can be seen that there is a significant selectivity for hydroxy tyrosol compared to the other components of the waste streams (see Table 2). The selectivity of hydroxy-tyrosol over gallic acid (ratios of OMTC) was 3.8. The use of NF concentrate of an olive oil waste stream gave improved membrane contactor unit performance with respect to flux, permeability and selectivity compared to using the membrane contactor to treat RO concentrate (Table 2).

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXPERIMENTAL SECTION

Reference is now made to the following examples, which together with the above descriptions, illustrate the invention in a non limiting fashion.

### Materials

NADIR® UF membranes having a Molecular Weight Cutoff (MWCO) of 50 kDA, were purchased from Microdyn-Nadir GmbH.

NF membrane, NF-270, was purchased from Dow Liquid separation systems and the membrane DK-5 was purchased from General Electric.

RO membranes used in concentration runs, RO-1 and RO-2 were Dow-Filmtec SW30-4040 four-inch spiral wound seawater reverse osmosis elements purchased from Dow Liquid separations.

Polypropylene (PP) non-woven membrane was purchased from AWA.

Polydimethylsiloxane (PDMS) membrane was prepared as described below.

PolyTriFluoropropylmethylSiloxane (PTFS) solution was purchased from Gelest.

Poly (4-vinylphenol) (P4VP, also known as PVP or polystyrene hydroxyl) was purchased from Sigma Aldrich.

Tetra ethyl ortho silicate (TEOS, crosslinker) was purchased from Sigma Aldrich.

Dibutyltin dilaurate (catalyst) was purchased from Sigma Aldrich.

All other chemicals were purchased from Sigma/Aldrich.

### General Synthesis Methods

### Membrane Manufacturing

The area of double (folded) support is 2×(10 cm×12 cm)=240 cm2.

Each support was washed overnight in 0.5% NaOH and then rinsed with DI water. Before use, the supports were placed in isopropanol to take out the water.

Before coating, the pores of supports are filled with a non crosslinked polysiloxane (MW 4200) which serves as a pore protector, to prevent compaction of the UF membrane during heating.

The coating solution was poured in a homemade metal bath, and the support was coated by standard dipping methods. After dipping, the coated support was drained at room temperature for 72 hours in a hood.

For the preparation of multilayered membranes, after each layer the coated support was drained in a hood for 30 minutes and cured in oven for 1 hour at 50° C. After curing the support was cooled for 15 minutes at room temperature.

### Coating Solution Preparation

Each of the components: Fluorinated polysiloxane Polytrifluoropropylmethylsiloxane (PTFS), Poly (4-vinylphenol) (PV4P), Tetraethylortosilicate, Dibutyltin dilaurate and Tyrosol, were weighed separately in small glasses with use of plastic pipettes. The solvent (THF) was added to obtain the desired concentration of each component.

Each component of the coating solution was dissolved separately at gentle mixing using a magnet stirrer at room temperature for 30 minutes.

The final coating solution was prepared as a mixture of the prescribed components in a closed glass container for 1 hour at room temperature.

Below are details for preparation of the membranes which include the preferable membrane formulations: #80 and #81

For membranes manufacturing the following solutions in large volumes:

A) PTFS 1% solution in THF: 1 g PTFS was dissolved in 99 ml of THF until full dissolution.

B) PV4P 1% solution in THF: 1 g PV4P was dissolved in 99 ml of THF until full dissolution.

C) 1 g dibutiltin dilaurate in 99 ml THF.

D) 1 ml TEOS in 99 ml THF.

E) 10 gr Tyrosol in 90 ml THF.

F) 0.5 g PTFS is dissolved in 99.5 ml THF.

G) 0.5 g PV4P is dissolved in 99.5 ml THF.

H) 5 gr Tyrosol is dissolved in 95 ml THF.

J) 0.5 ml dibutiltin dilaurate is dissolved in 99.5 ml THF.

K) 0.5 ml TEOS in 99.5 ml THF.

For each membrane, the various solutions of all the components were mixed, and the solvent (THF) was added to obtain a 10% solution for coating.

### Novel Membrane Contactor Unit Procedure

A system of membrane contactor units was designed and assembled to characterize the mass transfer properties of the membranes. These contactors were joined in series on the feed side so that all membranes in a given experiment were exposed to the same feed, and the contactors were arranged in parallel on the strip side so that there was a separate strip solution being cycled past each membrane contactor. This arrangement allowed either several different membranes to be tested, or replicates of the same membranes under identical operating conditions. The membrane contactor units were manufactured by use of 3D printer and Fused Deposition Modeling (FDM) Technology and had channel heights of approximately 2 mm and a membrane area of ˜21 cm² for each contactor.

After manufacturing, the tested membranes were rinsed in deionized water for 30 minutes and after this were placed in membrane contactor units with the selective layer facing to feed and were assembled with use of metal mounting hardware.

The pH of the stripping solution on the permeate side was manually maintained constant at pH=13 by adding NaOH solution. The samples were withdrawn from the feed and permeate phases at given time interval and sent for Total phenol and HPLC determination.

The characteristics and experimental conditions are provided below:

| Parameter | Value | |
|---|---|---|
| Feed circulation flow rate | 200 | ml/min |
| Stripping solution circulation | 200 | ml/min |
| flow rate | | |
| Feed concentration | 2-9 | gr/L |
| Stripping solution (NaOH at pH | 1 | gr/L |
| ~13) | | |
| Membrane materials | Polydimethylsiloxane, | |
| | PolyTriFluoropropylmethylSiloxane | |
| | Polyvinyl phenol | |
| | tetraethyl orthosilicate | |
| | dibutyltin dilaurate | |
| Membrane area | 3 cm × 7 cm = 21 cm² | |
| Volume of the feed tank | 1 | liter |
| Volume of the each permeate | 0.5 | liter |
| (stripping) solution tank | | |
| Temperature in the solutions | 25-50° | C. |

| | |
|---|---|
| pH in the permeate (stripping) | 11-13 |
| solution | |
| pH in the feed | 2-2.5 |

### Methods and Analysis

Percentages are weight percentages (wt), all fractions are by weight and all temperatures are in ° C., unless otherwise indicated.

Membrane performance was tested with respect to Mass flux (MF, in mg/m²*sec), Overall mass transfer coefficient (OMTC, in m/sec), stability and selectivity with respect to different solutes found in the feed stream.

The equation connecting OMTC to Mass flux was as follows:

OMTC=MF/(Average Feed Concentration of Polyphenol).

The following operating conditions were used: The feed was kept at pH 1-3 with HCl or H₂SO₄ and the permeate stream was kept at pH 11-13 with NaOH. If dialysis cells were used, then only mechanical stirring was employed for fixed volumes of feed and basic extractant solution located on opposite sides of the membranes. In the flow cells, feed was passed over the selective coated side of the membrane and the high pH strip solution was recycled over the backside of the composite membranes and served as the receiving phase for polyphenols and other organics which permeated the membrane from the feed solution.

### Membranes Characterization in Dialysis Cell

The test was made at average temperature of 25° C. The testing was carried out by placing the tested membrane in a dialysis cell. The tested sample covered the cell orifice with diameter 30 mm. Sample area was 706.5=². Each compartment volume was 50 ml.

The feed compartment was filled with synthetic mixtures of polyphenols. The pH of the feed solution was adjusted to pH=2 by H₂SO₄. The permeate compartment was filled by distilled water with pH=13 adjusted by NaOH.

At regular time intervals, samples from the permeate and from the feed sides of the cell were taken out and their Total phenol or HPL were analyzed. pH was not adjusted after each sample was taken due to its minimal change both in feed and in permeate compartments during 6 hours.

### Experimental Results

### Example 1: Preparation of Various PTFS-Coated Composite UF Membranes

An example of the membrane preparation method for one embodiment is as follows:

Poly (4-vinylphenol) (PV4P) was mixed at 40° C. for 1 hour with different amounts (see Table 1 for quantities) of tetra ethyl ortho silicate (TEOS, crosslinker). Then, the obtained mixture was added to a PolyTriFluoropropylmethylSiloxane (PTFS) solution with dibutyltin dilaurate catalyst (see Table 1 for quantities), mixed carefully again for 1 hour at room temperature and used immediately for the preparation of the composite membranes.

The obtained polymer solution was used as a coating solution in order to coat, by dipping, a thin film over a porous support (NADIR® UF membranes with MWCO=50 kDA), whereas the coating was done only on the upper selective side of the support membrane (without coating material being applied to the under, more porous, side). After preparation, the membranes were cured at 85° C. for 1 hour. After solvent evaporation and drying for several hours, the membrane could be used.

The characteristics of each of the new membranes are provided in Table 1 below. All permeabilities (OMTC) were calculated from Total Phenol data obtained in dialysis cell. All membranes in Table 1 used a 50 kDa UF support.

All the membranes that were made had a thickness of about 1-5 microns, based on polymer concentrations in the solutions and by setting a gap between the spreading blade and the membrane support to control the thickness of polymer solution coating the membrane support. The coating could be made thicker up to 10 microns.

TABLE 1

| | | dibutyltin | | | Overall |
|---|---|---|---|---|---|
| Sample # and | Polymer, ml, | dilaurate | TEOS Cross- | | mass |
| membrane | as 10% | Catalyst ml, | linker, ml, | Mass | transfer |
| composition | solution in | 1% solution | 5% solution | flux | coefficient, |
| (polymer type) | THF | in THF | in THF | mg/m² * sec | m/sec |
| #75. | 0.3 ml | 0.5 ml | 0.1 ml | 2.85 | 4.3 * 10⁻⁶ |
| PTFS 100% | | | | | |
| #76 | PTFS = 0.56 ml, | 0.4 ml | 0.07 ml | | |
| PTFS: PV4P | PV4P = 0.14 ml, | | | | |
| (8:2) | | | | | |
| #77 | PTFS = 0.42 ml, | 0.5 ml | 0.08 ml | | |
| 50 kDa | PV4P = 0.28 ml | | | | |
| PTFS:PV4P (6:4) | | | | | |
| #78 | PTFS = 0.56 ml,, | 0.4 ml | 0.07 ml | | |
| PTFS:PV4P (8:2) + | PV4P = 0.14 ml, | | | | |
| Tyrosol | Tyrosol = 0.5 m, | | | | |
| #79 | PTFS = 0.42 ml, | 0.5 ml | 0.08 ml | | |
| PTFS:PV4P (6:4) + | PV4P = 0.28 ml | | | | |
| Tyrosol | Tyrosol = 0.5 ml | | | | |
| #93 | 0.27:0.03 ml | 0.4 ml | 0.1 ml | 3.24 | 4.8 * 10⁻⁶ |
| PTFS:PV4P = 9:1 | | | | | |
| #94 | 0.24:0.06 ml | 0.5 ml | 0.2 ml | 3.67 | 3.85 * 10⁻⁶ |
| PTFS:PV4P = 8:2 | | | | | |
| #95 | 0.27:0.03 ml | 0.5 ml | 0.8 ml | 2.43 | 4.40 * 10⁻⁶ |
| PTFS:PV4P = 9:1 | | | | | |
| #97 | 0.18:0.12 ml | 0.8 ml | 0.6 ml | 2.84 | 5.06 * 10⁻⁶ |
| PTFS:PV4P = 6:4 | | | | | |
| #98 | 0.15:0.15 ml | 0.8 ml | 0.8 ml | 3.79 | 5.15 * 10⁻⁶ |
| PTFS:PV4P = 1:1 | | | | | |
| #99 | 0.15:0.15 ml | 0.8 ml | 0.8 ml | 3.1 | 4.62 * 10⁻⁶ |
| PTFS:PV4P = 1:1 | | | | | |
| without curing | | | | | |
| at 85° C. | | | | | |
| #100 | 0.3 ml | 0.5 ml | 0.1 ml | 4.25 * 10⁻² | 2.19 * 10⁻⁸ |
| PTFS 100% on PP | | | | | |
| Pure PP | | | | 1.2 * 10⁻¹ | 1.7 * 10⁻⁷ |
| nonwoven | | | | | |
| support | | | | | |
| UF 50 kDa | | | | 11.6 | 10.74 * 10⁻⁶ |

Table 2 shows the results of the a flow cell membrane contactor unit fitted with membrane #75, for extracting components of OMWW from NF or RO concentrates.

TABLE 2
Flux of polyphenols for different feeds treated
in membrane contactor with membrane #75.

| | | | | Overall |
|---|---|---|---|---|
| | | | Mass | mass |
| | Feed | Permeate | transfer | transfer |
| | components, | components, | flux, | coefficient, |
| Feed source | ppm | ppm | mg/m² * sec | m/sec |
| Concentrate after NF | Tyrosol 14.7 | Tyrosol 3.84 | 5.08 * 10⁻² | 3.46 * 10⁻⁶ |
| with use NF-270 at | | | | |
| 20 bar | | | | |
| Concentrate after NF | Hydroxytyrosol | Hydroxytyrosol | 1.59 * 10⁻² | 4.96 * 10⁻⁶ |
| with use NF-270 at | 3.2 | 1.2 | | |
| 20 bar | | | | |
| Concentrate after NF | Gallic Acid | Gallic Acid | 2.78 * 10⁻² | 1.34 * 10⁻⁶ |
| with use NF-270 at | 20.8 | 2.1 | | |
| 20 bar | | | | |
| Concentrate after | Tyrosol 565 | Tyrosol 9.6 | 1.27 * 10⁻¹ | 2.25 * 10⁻⁷ |
| reverse osmosis RO-1 | | | | |
| Concentrate after | Hydroxytyrosol | Hydroxytyrosol | 3.84 * 10⁻¹ | 4.5 * 10⁻⁷ |
| reverse osmosis RO-1 | 853 | 29 | | |
| Concentrate after | Gallic Acid 82 | Gallic Acid | 2.25 * 10⁻² | 2.74 * 10⁻⁷ |
| reverse osmosis RO-1 | | 1.7 | | |
| Concentrate after | Tyrosol 840 | Tyrosol 3.8 | 5.03 * 10⁻² | 5.98 * 10⁻⁸ |
| reverse osmosis RO-2 | | | | |
| Concentrate after | Hydroxytyrosol | Hydroxytyrosol | 1.07 * 10⁻¹ | 7.74 * 10⁻⁸ |
| reverse osmosis RO-2 | 1385 | 8.1 | | |
| Concentrate after | Gallic Acid 69 | Gallic Acid | 8.6 * 10⁻³ | 1.25 * 10⁻⁷ |
| reverse osmosis RO-2 | | 0.65 | | |

Table 3 shows the overall mass transfer coefficients for Membrane #76 (PTFS:PV4P=8:2) for different polyphenol components in a feed of NF concentrate. From this the selectivity can be calculated and shows that the membrane was most permeable to hydroxytyroxol followed by tyrosol followed by gallic acid.

TABLE 3

| | | | | Overall mass |
|---|---|---|---|---|
| | Feed | Permeate | | transfer |
| | components, | components, | Mass transfer | coefficient, |
| Feed source | ppm | ppm | flux, mg/m² * sec | m/sec |
| Concentrate after | Tyrosol 14.3 | Tyrosol 4.4 | 5.82 * 10⁻² | 4.07 * 10⁻⁶ |
| NF with use NF-270 | | | | |
| at 20 bar | | | | |
| Concentrate after | Hydroxytyrosol | Hydroxytyrosol | 2.38 * 10⁻² | 4.58 * 10⁻⁶ |
| NF with use NF-270 | 5.2 | 1.8 | | |
| at 20 bar | | | | |
| Concentrate after | Gallic Acid | Gallic Acid | 7.14 * 10⁻² | 3.88 * 10⁻⁶ |
| NF with use NF-270 | 18.4 | 5.4 | | |
| at 20 bar | | | | |

Table 4 shows the selectivity results for Membrane #77 (PDMS:PV4P=6:4)

TABLE 4

| | | | Mass | Overall mass |
|---|---|---|---|---|
| | Feed | Permeate | transfer | transfer |
| | components, | components, | flux, | coefficient, |
| Feed source | ppm | ppm | mg/m² * sec | m/sec |
| Concentrate | Tyrosol 13.6 | Tyrosol 4.7 | 6.22 * 10⁻² | 4.57 * 10⁻⁶ |
| after NF with | | | | |
| use NF-270 at | | | | |
| 20 bar | | | | |
| Concentrate | Hydroxy- | Hydroxy- | 2.25 * 10⁻² | 5.92 * 10⁻⁶ |
| after NF with | tyrosol 3.8 | tyrosol 1.7 | | |
| use NF-270 at | | | | |
| 20 bar | | | | |
| Concentrate | Gallic Acid | Gallic Acid | 3.31 * 10⁻² | 1.48 * 10⁻⁶ |
| after NF with | 22.4 | 2.5 | | |
| use NF-270 at | | | | |
| 20 bar | | | | |

Table 5 shows the effect of selective membrane crosslinker concentration on composite Membranes (for a 50K UF support) in terms of phenol flux and Salt rejection (1000 ppm NaCl).

TABLE 5

| | Cross-linker | | | |
|---|---|---|---|---|
| Selective layer | TEOS, 0.1% | Mass | | |
| polymer | in n- | flux, mg/m2, | OMTC, | Na |
| composition | hexane, ml | sec, ×10⁻³ | m/sec, ×10⁻⁴ | rejection, % |
| #42 PDMS-2 | 0.2 | 9.6 | 1.05 | 61.4 |
| #43 PDMS-2 | 0.4 | 8.6 | 0.94 | 66.1 |
| #44 PDMS-1 + | 0.3 | 9.8 | 1.08 | 57.5 |
| PTFS = 1:10 | | | | |
| #45 PDMS-2 | 0.6 | 6.1 | 0.65 | 80.3 |
| #47 PDMS-1 + | 0.4 | 6.4 | 0.68 | 74.4 |
| PTFS = 2:8 | | | | |
| #48 PDMS-2 | 0.8 | 5.8 | 0.62 | 93.8 |
| #50 PDMS-1 + | 0.7 | 7.6 | 0.82 | 91.8 |
| PTFS = 4:6 | | | | |
| Dana's data | Not available | 2.38 | 0.26 | Not available |
| Livingston data | Not available | Not | 0.31 | Not available |
| | | available | | |

In another experiment, after 30 hours of operation the tyrosol/coumaric selectivity factor (Cp/Cf)tyrosol/(Cp/Cf)coumaric increased from 1.15 for a pure PTFS membrane (#75) to 2.42 for a PTFS:PV4P membrane (#97, PTFS:PV4P/6:4).

### Example 2: Preparation of Composite Membranes Using Different Support Membranes

Membrane 94 (PTFS:PV4P 8:2) was prepared as described in Example 1 using different supports upon which the selective layer was coated: These supports included UF membranes of 50 kDa, 100 kDa, 150 kDa, and 300 kDa MWCO whose polymer matrices are based on stable engineering plastics such as polyethersulfone and polysulfone.

FIG. 2 discloses the effect of UF support on PTFS composite membrane permeability of phenol. The selective PTFS layer thickness was estimated as 1-3 μm. FIG. 2 shows that the composite membranes on the more open UF supports (namely, having a higher MW cutoff) had higher mass flux and OMTC than the tighter UF membranes (having a lower MW cutoff).

### Example 3: Preparation of Multilayered Membranes

In this example membranes using similar chemistry to what is described in the previous examples were used to make multilayered membranes.

The composition of several multi-layered membranes is presented in Table 6 below.

Membrane 80 is an example of a double layered selective membrane on a UF support, wherein the first layer on the UF support is PV4P with tyrosol, followed by a layer comprising PTFS:PV4P (6:4).

Membrane 81 is an example of a triple layer wherein the first layer on the UF support is PTFS:PV4P (6:4). The middle layer membrane is of PV4-P with tyrosol, followed by a top layer of PTFS:PV4P (6:4).

TABLE 6

| | | Dibutyltin | Tetraethyl | | |
|---|---|---|---|---|---|
| Membrane | | dilaurate, | orthosilicate, | | Polymer |
| # and | | catalyst, | crosslinker, | Coating | solution |
| support | Polymer | ml | ml | solvent | concentration, % |
| #80 | 1^{st}layer: | 0.2 | 0.035 | THF | 5 |
| New double | PV4P = 0.7 ml, | | | | |
| layer | Tyrosol = 0.2 ml | | | | |
| membrane | 2^{nd} layer: | 0.2 | 0.035 | THF | 5 |
| 50 kDa | PTFS 0.42 ml, | | | | |
| | PV4P = 0.28 ml | | | | |
| | (6:4) | | | | |
| #81 | 1^{st} layer: | 0.2 | 0.035 | THF | 5 |
| New multi- | PTFS = 0.42 ml, | | | | |
| layer | PV4P = 0.28 ml | | | | |
| membrane | (6:4), | | | | |
| 50 kDa | 2^{nd} layer: | 0.2 | 0.035 | THF | 5 |
| | PV4P = 0.7 ml, | | | | |
| | Tyrosol = 0.2 ml | | | | |
| | 3^{rd} top layer: | 0.2 | 0.035 | THF | 5 |
| | PTFS 0.42 ml, | | | | |
| | PV4P = 0.28 ml | | | | |
| | (6:4) | | | | |

The Results of operating two membrane contactors fitted with different membranes on a common feed, which was an NF concentrate, are presented in Table 7, showing the selectivity of a bi-layered membrane (#80), in comparison to a single-layered membrane (#77). In this experiment the contactor was operated at 50° C. and the analysis of the recycling permeate (strip) solution composition was done after 24 hours of continuous operation of the membrane contactor.

TABLE 7

| | Hydroxytyrosol | | Ratio (%) |
|---|---|---|---|
| Stream | and Tyrosol | TOC | hydroxytyrosol + Tyrosol |
| descriptor | g/L | g/L | of TOC |
| Feed (start -> | 13.3 --> 6.6 | 65 --> 40 | 12.8 -->10.4 |
| finish) | | | |
| Permeate from | 0.425 | 1.21 | 21.9 |
| Membrane 77 | | | |
| Single layer | | | |
| Permeate from | 0.537 | 1.32 | 25.3 |
| Membrane 80 | | | |
| bi-layer | | | |

### Example 4: Biodegradation of Olive Oil Mill Permeate in Batch Mode Operation

This example describes the biodegradability of the NF permeate obtained after removing the polyphenols concentrate, namely the permeate number 9 of the scheme presented in FIG. 5.
1. Preparation of olive oil mill permeate in two different dilutions (1:5 and 1:10) with and kept in two 1 L Erlenmeyer flask. These dilutions represent dilution of NF OMW permeate with standard domestic wastewater stream fed to a wastewater treatment plant. The MBR sludge was added to each of the flask and the initial VSS concentration was measured to be ˜0.5 g/L in both the flasks.
2. The pH was maintained near neutral by adding calcium hydroxide solution.
3. The flasks were kept in a shaker water bath and the temperature was maintained at ˜27° C.
4. The air was pumped into the flasks through stone diffusers
5. The samples from both the flasks were taken at different time intervals and the reduction in the DOC concentration with time was measured.

Results:

| Time | | |
|---|---|---|
| (hours) | DOC (mg/l) 1:5 | DOC (mg/l) 1:10 |
| 0 | 648 | 404.8 |
| 24 | 131.4 | 41.6 |
| 48 | 74.8 | 34 |
| 68 | 52.7 | 20.8 |
| 97 | 49.5 | 18.8 |
| 120 | 33.2 | 18 |
| 144 | 20 | 16.4 |

1-33. (canceled)

34. A stable composite membrane comprising a porous support having one or more thin selective layers coated on a top surface thereof, whereas at least one of said thin selective layers comprises a crosslinked fluorinated silicone polymer, and further wherein the total thickness of said one or more thin selective layers ranges between 0.1 to 10 microns.

35. The composite membrane of claim 34, wherein the total thickness of said one or more thin selective layers ranges between 1 to 5 microns.

36. The composite membrane of claim 34, wherein one or more of said thin selective layers further comprises at least one polyphenol and/or at least one polymer having one or more aromatic hydroxyl groups.

37. The composite membrane of claim 36, wherein said polyphenol is selected from polyvinyl phenol and/or hydroxy tyrosol.

38. The composite membrane of claim 34 wherein said fluorinated silicone polymer is selected from fluorinated polysiloxanes, fluorinated polysilanes, fluorinated chlorosilanes, fluorinated alkoxysilanes, fluorinated aminosilanes, fluorinated silicone esters, fluorinated polydialkylsiloxanes, and phenyl substituted fluorinated polysiloxanes.

39. The composite membrane of claim 34, wherein said porous support is selected from an ultrafiltration (UF) membrane, a microfiltration membrane (MF) and a non-woven polymer.

40. The composite membrane of claim 34, wherein said one or more thin selective layers further comprises a non-fluorinated silicone polymer.

41. The composite membrane of claim 34, wherein one or more of said thin selective layers further comprises a monophenol.

42. The composite membrane of claim 34, comprising a single thin selective layer.

43. The composite membrane of claim 42, wherein said single thin selective layer comprises Poly-trifluoropropylmethylSiloxane (PTFS) and polyvinyl phenol.

44. The composite membrane of claim 43, further comprising Tyrosol.

45. The composite membrane of claim 34, comprising two selective layers.

46. The composite membrane of claim 45, wherein one of said layers comprises crosslinked Poly-trifluoropropylmethylSiloxane (PTFS) and polyvinyl phenol, and a second of said layers comprises polyvinyl phenol and tyrosol.

47. The composite membrane of claim 34, comprising three selective layers.

48. The composite membrane of claim 47, wherein both a first and a last of said layers comprises a crosslinked Poly-trifluoropropylmethylSiloxane (PTFS), and whereas a second of said layers, in between said first and said last layers, comprises polyvinyl phenol and tyrosol.

49. A process for the preparation of the composite membrane of claim 34, said process comprising
a. Preparing a first coating solution comprising a crosslinkable fluorinated silicone polymer, a crosslinking agent, a catalyst and a solvent, and optionally a polyphenol and/or a monophenol;
b. contacting said coating solution onto a top surface of a porous support thereby forming a layer onto said support;
c. curing said layer for a time ranging from 30 minutes to 4000 minutes and at a temperature ranging from 20° C. to 85° C. to obtain a first stable thin selective layer having a thickness ranging between 0.1 to 10 microns; and
d. optionally further preparing one or more additional coating solutions, each comprising one or more of a crosslinkable fluorinated silicone polymer, a fluorinated silicone polymer, a non-fluorinated silicone polymer, a crosslinking agent, a catalyst, a polyphenol, a monophenol, and a solvent; contacting said one or more additional coating solutions with said first thin selective layer, and curing said additional layers, so as to obtain a total thickness of said one or more thin selective layers ranging between 0.1 to 10 microns.

50. The process of claim 49, wherein said crosslinking agent is selected from: organic peroxides, inorganic peroxides, alkoxysilane, and a polysiloxane.

51. A membrane contactor unit, comprising the composite membrane of claim 34, in which the selective side of said membrane faces a feed stream rich in polyphenols whereas the porous side of said membrane is adjacent to a high pH strip solution.

52. A process for obtaining a polyphenol rich concentrate of an olive oil mill wastewater stream, said process comprising:
a. Contacting an olive oil mill wastewater stream with an acid, to obtain an acidified olive oil mill wastewater stream at a pH ranging from 2 to 2.5;
b. Feeding said acidified olive oil mill wastewater stream into a holding tank, and phase separating from said olive oil mill wastewater:
i. a bottom layer stream rich in suspended solids,
ii. a top layer stream rich in olive oil
iii. a middle layer stream, that is largely devoid of suspended solids and of olive oil;
c. Feeding said middle layer stream into an ultrafiltration unit (UF), thereby separating said middle layer stream into a UF permeate and a UF concentrate;
d. Feeding said UF permeate into a nanofiltration unit (NF), thereby separating said UF permeate into a NF concentrate rich in polyphenols and a NF permeate largely free of polyphenols;
e. Separately feeding each of said UF concentrate and said NF concentrate into a selective side of a membrane contactor unit comprising the composite membrane of claim 1, and circulating said concentrate next to a selective side of said membrane, further whereas a high pH strip solution is circulated next to a porous side of said membrane, to obtain a polyphenol rich permeate stream at the porous side of the contactor membrane unit;
f. passing said polyphenol rich permeate stream through a second nanofiltration unit (NF2), thereby obtaining a concentrate which is a polyphenol rich product, and a caustic solution permeate.

53. The process of claim 52, wherein said polyphenol rich product comprises at least 5% of phenols and/or polyphenols.

54. The process of claim 52, further comprising purifying said polyphenol rich product of NF2.

55. The process of claim 52, further comprising passing said first NF permeate through a biological treatment unit, to obtain an irrigation-adequate stream having a chemical oxygen demand (COD) lower than 300 mg/L.

56. The process of claim 52, further comprising recycling said caustic solution permeate into said porous side of said membrane contactor unit, thereby stripping out additional polyphenol.
